(19) United States
(12) Patent Application Publication
(10) Pub. No.: US 2016/0324582 A1
(43) Pub. Date: Nov. 10, 2016

(54) METHODS FOR THE NONINVASIVE DETERMINATION OF PERFUSION, BLOOD FLOW, AND CAPILLARITY

(71) Applicant: Ghassan S. Kassab, La Jolla, CA (US)

(72) Inventor: Ghassan S. Kassab, La Jolla, CA (US)

(21) Appl. No.: 15/212,043

(22) Filed: Jul. 15, 2016

Related U.S. Application Data
(63) Continuation-in-part of application No. 15/168,807, filed on May 31, 2016.
Application No. 15/168,807 is a continuation-in-part of application No. 13/106,027, filed on May 12, 2011.
Application No. 13/106,027 is a continuation-in-part of application No. 12/864,016, filed on Jul. 22, 2010, now Pat. No. 8,670,943.
Application No. 15/168,807 is a continuation-in-part of application No. 14/205,035, filed on Mar. 11, 2014.
Application No. 14/205,035 is a continuation of application No. 12/864,016, filed on Jul. 22, 2010, now Pat. No. 8,670,943.
(60) Provisional application No. 62/192,952, filed on Jul. 15, 2015.
(60) Provisional application No. 60/881,833, filed on Jan. 23, 2007.

(51) Int. Cl. A61B 34/10; A61B 10/02; G06F 17/50; G06T 7/00; G06T 7/40; G06F 17/11; A61B 5/026; A61B 5/00

(52) U.S. Cl. CPC A61B 34/10; A61B 5/026; A61B 10/02; A61B 5/4839; G06T 7/0014; G06T 7/403; G06F 17/11; G06F 17/5009; A61B 2034/105; A61B 2034/108; G06T 2207/30104

(57) ABSTRACT

A framework for the accurate and noninvasive determination of perfusion in a mammal is provided, including a novel scaling law of a form-function relationship between the number of capillaries in a vascular network as compared to the perfusion of such network. Methods are disclosed that apply such scaling laws in connection with the steps of determining a capillary density of a targeted tissue comprising at least a portion of a capillary network, and calculating perfusion of the targeted tissue based on the determined capillary density of the targeted tissue. Additional methods for determining a therapeutic drug dosage for a biological subject are also provided based on the scaling-laws hereof, as well as methods of identifying a deviation in perfusion rates in a mammal noninvasively.

PRIORITY

This application a) is related to, and claims the priority benefit of, U.S. Provisional Patent Application Ser. No. 62/192,952, filed Jul. 15, 2015, b) is related to, claims the priority benefit of, and is a U.S. continuation-in-part patent application of, U.S. patent application Ser. No. 15/168,807, filed May 31, 2016, which is related to, claims the priority benefit of, and is a U.S. continuation-in-part patent application of, U.S. patent application Ser. No. 13/106,027, filed May 12, 2011, which is related to, claims the priority benefit of, and is a U.S. continuation-in-part patent application of, U.S. patent application Ser. No. 12/864,016, filed Jul. 22, 2010 and issued as U.S. Pat. No. 8,670,943 on Mar. 11, 2014, which is related to, claims the priority benefit of, and is a U.S. Section 371 national stage patent application of, International Patent Application Serial No. PCT/US2008/072925, filed Aug. 12, 2008, which is related to, claims the priority benefit of, and is an international continuation-in-part application of, International Patent Application Serial No. PCT/US2008/000762, filed Jan. 22, 2008, which is related to, and claims the priority benefit of, U.S. Provisional Patent Application Ser. No. 60/881,833, filed Jan. 23, 2007, and c) is related to, claims the priority benefit of, and is a U.S. continuation-in-part patent application of, U.S. patent application Ser. No. 14/205,035, filed Mar. 11, 2014, which is related to, claims the priority benefit of, and is a U.S. continuation patent application of, U.S. patent application Ser. No. 12/864,016, filed Jul. 22, 2010 and issued as U.S. Pat. No. 8,670,943 on Mar. 11, 2014, which is related to, claims the priority benefit of, and is a U.S. Section 371 national stage patent application of, International Patent Application Serial No. PCT/US2008/072925, filed Aug. 12, 2008, which is related to, claims the priority benefit of, and is an international continuation-in-part application of, International Patent Application Serial No. PCT/US2008/000762, filed Jan. 22, 2008, which is related to, and claims the priority benefit of, U.S. Provisional Patent Application Ser. No. 60/881,833, filed Jan. 23, 2007. The contents of each of the foregoing applications and patent are hereby incorporated by reference in their entireties into this disclosure.

### BACKGROUND

The major role of vascular networks in the circulatory system is to transport blood, oxygen, nutrients, hormones, and cellular waste to and from various organs to maintain biological homeostasis. Indeed, physiological vascular trees provide flow transport to the capillary network to support tissue demands. Adequate perfusion (volumetric blood flow per unit mass of tissue) through a vascular transport structure—i.e. perfusion that satisfies the metabolic requirements of an organ or tissue—is essential for any organ or tissue, irrespective of species. Indeed, too low of perfusion can cause hypoxia, ischemia, cell death and, ultimately, the loss of organ or tissue function.

In light of this, vascular development is generally guided by tissue metabolic needs. The number density of capillaries can be determined from histological sections of biopsy specimens of animals and patients. In certain cases, for example, the number density of capillaries may be seen to increase in tumors in accordance with an increase in blood flow to enhance growth of the tissue. Alternatively, the number density of capillaries can also be used to identify patho-physiology such as where the number density of capillaries is decreased due to an infarct, or in hypertension or obesity, etc. (which may eventually lead to malnutrition, atrophy and/or death of the tissue). While the capillarity of a tissue has an effect on the degree to which such tissue is perfused, a quantifiable and accurate relationship between the two is not conventionally known.

Accurately measuring perfusion can be a useful tool for providing information about tissue viability and health, as well as in distinguishing the border between physiology and path-physiology of a tissue or organ. Conventionally, nuclear imaging has been used to obtain perfusion measurements, but this is unfortunately an expensive and non-routinely employed modality. Additionally, histological assessment of biopsy tissue, including capillary density measurements, are more common, but invasive. Furthermore, such histological assessments' connection with flow—and hence function—is empirical and qualitative at best.

Especially considering that the early detection of perfusion problems is beneficial for proper diagnosis and effective treatment, it would be beneficial to provide a framework for an accurate and non-invasive way to determine the blood flow that perfuses a capillary network. Such a framework may include, for example, a novel scaling law of a form-function relation between the number of capillaries in a vascular network as compared to the perfusion of such network.

### BRIEF SUMMARY

In at least one exemplary embodiment of a method for determining perfusion in a mammal, the method comprises the steps of: determining a capillary density of a targeted tissue comprising at least a portion of a capillary network, the capillary network being part of a mammalian biological tree; and calculating perfusion of the targeted tissue based on the determined capillary density of the targeted tissue. The calculated perfusion may be linearly proportional to the capillary density. Still further embodiments of the method may additionally include the step of engineering an artificial tissue comprising a vascular network based on the calculated perfusion. In at least one optional embodiment, the method may further comprise the step of calculating total flow into the mammalian biological tree based on the determined capillary density of the targeted tissue. There, in at least one exemplary embodiment, such calculated total flow may be linearly proportional to the capillary density.

In additional embodiments of the method, the step of determining a capillary density of a targeted tissue comprises the steps of obtaining a biopsy specimen from the mammal and determining the capillary density of the targeted tissue histologically, wherein the biopsy specimen is representative of the targeted tissue.

In other embodiments of the method, the step of determining a capillary density of a targeted tissue may comprise the steps of: using a processor to produce an image showing at least part of the mammalian biological tree proximal to the capillary network of the targeted tissue, wherein the processor is operably connected to a storage medium capable of receiving and storing the image; identifying a crown length of a vessel portion from the mammalian biological tree image; and calculating the capillary density of the targeted tissue based on the crown length of the vessel portion.

The present disclosure also describes methods for determining a therapeutic drug dosage for a biological subject of a first species. In at least one exemplary example of such a method, the method comprises the steps of: determining a capillary density of a targeted tissue, the targeted tissue comprising at least a portion of a capillary network that is part of a mammalian biological tree; calculating the perfusion of the targeted tissue based on the determined capillary density of the targeted tissue; and titrating a proper dosage of a therapeutic drug based on the calculated perfusion of the targeted tissue. There, the calculated perfusion of the targeted tissue may be linearly proportional to the capillary density of the targeted tissue.

In at least one embodiment of the method, the step of determining a capillary density of a targeted tissue may be performed histologically. For example, in at least one embodiment, the step of determining a capillary density of a targeted tissue may comprise the steps of obtaining a biopsy specimen from the biological test subject, and determining the capillary density of the targeted tissue histologically, with the biopsy specimen being representative of the targeted tissue of the biological test subject.

In yet another embodiment of the method, the targeted tissue used in the method may be a tissue of a biological test subject of a second species, as opposed to the tissue of the biological subject for which the therapeutic drug dosage is to be calculated. By way of a non-limiting example, the first species of the biological subject may comprise a human and the second species of the biological test subject may be selected from a group consisting of a rodent and a pig. In such embodiments, the method may further comprise the steps of: scaling the proper dosage of the therapeutic drug from the second species to the first species; and normalizing the scaled dosage with respect to a weight of the biological subject of the first species. Still further, in at least one alternative embodiment, instead of calculating the perfusion of the targeted tissue of the biological test subject of the second species, the method may comprise the step of calculating total flow into the mammalian biological tree based on the determined capillary density of the targeted tissue of the biological test subject, and the step of titrating a proper dosage of a therapeutic drug for a biological subject of a second species may be based on the calculated total flow of the targeted tissue of the biological test subject. In such embodiments, the calculated total flow may be linearly proportional to the capillary density.

Methods of identifying a deviation in perfusion rates in a mammal are also provided. At least one exemplary embodiment of such a method comprises the steps of: establishing a baseline capillary density from a plurality of healthy mammals of a first species; establishing a calculated baseline perfusion rate based on the baseline capillary density; determining a capillary density of a test subject; and comparing the capillary density of the test subject to the baseline capillary density. There, a deviation from the baseline capillary density is indicative of the test subject having a perfusion rate that is not in accordance with the baseline perfusion rate. The test subject may comprise a mammal of a second species—for example, and without limitation, a human. Furthermore, in at least one embodiment, the calculated baseline perfusion rate is linearly proportional to the baseline capillary density.

In other embodiments of the method for identifying a deviation in perfusion rates in a mammal, the method may further comprise the steps of: identifying that the capillary density of the test subject deviates from the baseline capillary density; calculating a perfusion rate of a test subject based on the capillary density of the test subject; and assigning a diagnosis to the test subject based on the calculated perfusion rate, with the calculated perfusion rate of the test subject being linearly proportional to the capillary density of the test subject.

In certain embodiments of the method, the step of determining a capillary density of a test subject may be performed histologically. Alternatively, the step of determining a capillary density of a test subject may be performed using an image of a capillary network that is part of a biological tree of the test subject.

### BRIEF DESCRIPTION OF THE DRAWINGS

FIG. 1 shows an illustration of a definition of a stem-crown unit according to at least one embodiment of the present disclosure;

FIGS. 2A-2C show the intraspecific relation between normalized stem flow and normalized number of capillaries belonging to the stem for the RCA (FIG. 2A), LAD (FIG. 2B), and LCx (FIG. 2C) arterial trees, according to at least one embodiment of the present disclosure;

FIGS. 3A and 3B show the intraspecific relationship between 1) stem flow and number of capillaries (FIG. 3A), and 2) normalized stem flow and normalized number of capillaries for various organs and species (FIG. 3B) as denoted by the following symbols:

| | | | | |
|---|---|---|---|---|
| ♦ Hamster Muscle | ♦ Pig RCA | Δ Cat Lungs (venous) | ▴ Human Conjuctive (arterial) | ⋄ Human Lungs III (arterial) |
| ♦ Rat Lungs | ▴ Pig LAD | □ Cat Sartorius Muscle (control) | ▪ Human Conjunctive (venous) | Δ Human Lungs IV (venous) |
| ▴ Rat Mesentary | □ Pig LCX | ∘ Cat Sartorius Muscle (vasodilation) | Human Lungs I (arterial) | □ Human Lungs V (venous) |
| ♦ Rabbit Omentum | [symbol] Cat Lungs (arterial) | ♦ Human Skeletal Muscle | x Human Lungs II (arterial); | |

and

FIG. 4 shows the interspecific relationship between inlet stem flow and total number of capillaries for various organs and species as denoted by the following symbols (y=8.05*10⁻⁷×^{1.18.},R²=0.910):

♦ Hamster Muscle
♦ Rat Lungs
▴ Rat Mesentery
♦ Rabbit Omentum
♦ Pig RCA
▴ Pig LAD
▪ Pig LCX
⋄ Cat Lungs (arterial)
Δ Cat Lungs (venous)
□ Cat Sartorius Muscle (control)
◯ Cat Sartorius Muscle (vasodilation)
♦ Human Skeletal Muscle
▴ Human Conjunctiva (arterial)
▪ Human Conjuctiva (venous)
 Human Lungs I (arterial)
X Human Lungs II (arterial)
⋄ Human Lungs III (arterial)
Δ Human Lungs IV (venous)
□ Human Lungs V (venous);

FIG. 5 shows a diagnostic system and/or a data computation system according to at least one embodiment of the present disclosure;

FIG. 6A shows a data computation system according to at least one exemplary embodiment of the present disclosure; and

FIG. 6B shows an exemplary embodiment of a data computation device according to at least one embodiment of the present disclosure.

### DETAILED DESCRIPTION

The disclosure of the present application provides a framework for an analytical determination of blood flow or perfusion (volumetric blood flow per unit of mass of tissue) from the number of capillaries in a vascular network of a biopsy specimen. Perhaps more specifically, the present disclosure provides novel scaling laws related to the form-function relationship between the number of capillaries in a vascular network and the blood flow that perfuses such network. Such scaling laws are inter-specific (i.e. across various species including rats, cats, rabbits, pigs, hamsters, and humans), and were validated in intra-specific vascular trees (e.g., coronary, pulmonary, mesenteric vessels, skeletal muscle vasculature, and conjunctiva vessels) for which there exists morphometric data, thus demonstrating their accuracy and ease of use. Additionally, the present scaling laws are further supported by nature's proportionality law between the flow needed to nourish an organ and the number of capillaries needed to distribute such flow to the tissue of the organism.

For the purposes of promoting an understanding of the principles of the present disclosure, reference will now be made to the embodiments illustrated in the drawings, and specific language will be used to describe the same. It will nevertheless be understood that no limitation of the scope of the present disclosure is thereby intended.

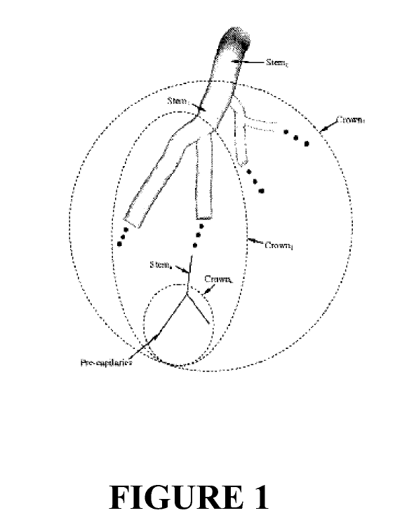

Several concepts are defined to formulate scaling laws of the disclosure of the present application. FIG. 1 shows a schematic illustration of the definition of the stem-crown unit. A vessel segment is defined as a “stem” and the entire tree distal to the stem is defined as a “crown,” as shown in FIG. 1. At each bifurcation, there is a unique stem-crown unit. Three stem-crown units are shown successively in FIG. 1 (1, 2, and n), with the smallest unit corresponding to an arteriole-capillary (for an arterial tree) or venule-capillary unit (for a venous tree). An entire vascular tree, or substantially the entire vascular tree, consists of many stem-crown units down to, for example, the smallest arteriole- or venule-capillary units. Functionally, each stem supplies or collects blood from the crown for an arterial or venous tree, respectively. The present analysis applies strictly to a tree structure (arterial or venous) down to the first capillary bifurcation.

One of the oldest hypotheses in biology and medicine is the structure-function relation. This hypothesis states that, in biological organisms, structural design is matched to functional demand. In other words, form serves function and function influences form. Living organisms show a remarkable variety of structures and sizes. Despite this heterogeneity and complexity, many of the most fundamental biologic processes manifest an extraordinary simplicity when viewed as a function of size. Allometric scaling laws describe how biologic parameters vary with scale, regardless of the differences among the organisms. Scaling laws arise from common underlying mechanisms that are independent of the specific nature of individual organisms. In particular, hierarchical fractal-like branching networks—which distribute energy and materials—are considered to play a central role. Although a number of scaling relations relating structure to function have been previously validated (e.g., flow-diameter and flow-length), to date an equivalent relation between flow and capillarity (i.e. the number or density of capillaries) has not been found. Here, novel scaling relations between diameter and flow and a conservation of mass principle, in conjunction with the relative uniformity of capillary dimensions, provides yet another link between structure (capillary numbers) and function (blood flow).

The novel scaling laws hereof demonstrate a novel form-function relation between the number of capillaries in a vascular network and the blood flow that perfuses such network. Additionally, since the structure-function relation is pervasive in biology, the novel scaling laws of the present disclosure can also be used to demonstrate a direct relationship between flow through a branch (i.e. stem flow) of an organ vascular system and the respective number of capillaries through which the blood flow distributes.

These scaling laws were derived based on the following axioms: 1) conservation of mass, 2) scaling law relationships between flow through and diameter of a capillary vessel, and 3) the relative uniformity of diameter of arterial capillaries, and have been validated based on existing data of a given organ in a given species (intraspecific) and across a number of various species (interspecific). The power-law scaling relation between flow and diameter is well-established and conventionally known as Murry's law. Although the exponent of the flow-diameter relation taken as 3 by Murry has been long debated and even disproven for certain organs, the power-law form itself has not been contested and is universally accepted as a reflection of the optimized design of the vascular system. Accordingly, the second axiom for the novel flow-capillarity laws hereof is well rooted.

Additionally, the relative uniformity of the diameter of arterial capillaries has been previously shown by Kassab and Fung for the coronary vasculature. See G.S. Kassab and Y. C. Fung, Topology and dimensions of the pig coronary capillary network. Am. J Physiol. Heart Circ. Physiol. 267 (1 pt 2): H319-H325, 1994, which is hereby incorporated herein by reference in its entirety. The coefficient of variation (CV=SD/Mean) is about 0.15 and about 0.18 for right and left ventricle walls, respectively. Furthermore, it is well recognized that the capillary dimensions are generally conserved across species (e.g., capillary diameters are similar in mice and elephants).

As previously stated, the novel formulation hereof invokes the law of conservation of mass, which requires the flow at the inlet of the tree or crown (Q^{s} stem flow) to be equal to the sum of the flows at the first capillary segment, Q^{c}; namely:

$${Q}^{s} = {\sum }_{i = 1}^{N} {Q}_{i}^{c} \qquad [ 1 ]$$

where N is the number of capillaries perfused by a given stem. Relation between flow and diameter has been previously determined as Q=K_{QD}D^{δ}. As such, Eq. [1] can be rewritten as:

$${Q}^{s} = {\sum }_{i = 1}^{N} {( {D}^{c} )}_{i}^{δ} \qquad [ 2 ]$$

Assuming that the diameters of the first segment of capillaries are approximately uniform and given by D̅_{c}, Eq. [2] reduces to:

Q^{s}≅kN_{c} [3]

where k=K_{QD}D̅_{c}^{δ} and is approximately constant. Accordingly, this demonstrates that the inlet flow is proportional to the total number of capillary vessels. Normalizing the flow and capillarity with respect to an entire tree obtains the following:

$$\frac{{Q}_{s}}{{Q}_{s , ma x}} = {( \frac{{N}_{c}}{{N}_{c , ma x}} )}^{λ} \qquad [ 4 ]$$

where Q_{s,max} and N_{c,max} are the inlet flow and the total number of capillaries in the vascular system, respectively. As provided herein, it was tested that λ is equal to 1 and, hence, the form of Eq. [4] is equivalent to that of Eq. [3] for various vascular trees.

The novel scaling laws hereof (and the related validation data provided below) demonstrate that the total blood flow into a vascular network is linearly proportional to the number of capillaries that distribute such flow. This relation has been found to hold true not only within a single species for a particular vascular network (intraspecific), but also across species (interspecific). Indeed, as provided herein, the novel scaling laws were validated for numerous vascular networks not only between the same species, but also across various species for which anatomical data exists in the literature in both detailed asymmetric networks as well as simplified symmetric networks.

Note that blood flow is directly related to perfusion, which is expressed as flow per mass. Accordingly, like blood flow, perfusion also relates proportionally to the number of capillaries per mass according to Eq. [3]. As mass is proportional to the volume of tissue through density, the perfusion is directly related to the number of capillaries per volume of tissue or number density as can be determined histologically or otherwise. In this manner, the linear scaling laws expressed in Eq. [3] allow for a direct connection between structure (number density) and function (perfusion).

The scaling laws disclosed herein have several clinical diagnostic implications. Namely, these scaling laws can be used to easily and noninvasively identify tissue pathology and/or suboptimal perfusion of a tissue or organ. As previously discussed, adequate perfusion (volumetric flow per mass of tissue) is essential for any organ or tissue because it directly affects the organ's/tissue's health and function. Simply by determining a functional capillary density of a targeted tissue, a practitioner can accurately calculate the perfusion and/or blood flow to such tissue without employing conventional invasive modalities.

The functional capillary density of a targeted tissue can be determined in several ways. It will be appreciated that the approaches described herein are significantly less invasive than the currently available conventional procedures associated with determining perfusion and/or blood flow.

In at least one embodiment, the determination of functional capillary density can be performed histologically (e.g. through the histological study of biopsy specimens in vitro). For example, a biopsy specimen of a targeted tissue or organ may be extracted and histologically prepared to count the number density of capillaries therein (e.g., number of capillaries per mm²). Pursuant to the disclosed scaling laws, the determined number density of capillaries of the specimen can then be used to indicate the flow or perfusion (flow per mass). Furthermore, the scaling laws hereof can also be used to will effectively scale the data determined from the biopsy specimen to a desired portion of the vascular network.

Alternatively, the functional capillary density may be calculated based on certain dimensions of the relevant vascular network. There, standard clinical imaging of blood vessel anatomy can be used in conjunction with the novel scaling laws hereof to yield functional data on perfusion, tissue health, and/or capillarity. Perhaps more specifically, the linearity identified by the novel scaling laws hereof between stem flow and crown length in a truncated tree model supports that the crown length is linearly related to the number of capillaries present within a targeted tissue/organ. As such, the functional capillary density can be calculated through application of the novel scaling laws hereof from the length of a vascular network, which can be obtained non-invasively through standard medical imaging. For example, in at least one exemplary embodiment, the length of the desired portion of the vascular network (e.g., the crown length) may be obtained from magnetic resonance imaging (or like imaging modalities) using the systems, methods, and techniques set forth in U.S. patent application Ser. No. 13/106,035 to Kassab et al., which is hereby incorporated by reference herein in its entirety.

The scaling laws provided herein have numerous applications. As previously noted, clinically, the scaling laws can be used in diagnosis to easily and noninvasively identify tissue pathology and/or suboptimal perfusion of a tissue or organ. For example, a capillary density can be established for “normal” subjects (e.g., volunteers) and the corresponding perfusion and/or blood flow rate(s) can be calculated therefrom. This established baseline can then be used as a barometer to easily identify changes in the number density (and thus perfusion) in patients. For example, a patient having a capillary density that falls outside of the established baseline would then be diagnosed to reflect the expected change in perfusion (similar to blood pressure measurements, etc.).

The scaling laws disclosed herein also have applications with respect to the tissue engineering of vascular networks. The scaling laws hereof can serve as a biomimetic principle for creating vascularized artificial tissues to carry out the function of perfusion. Similarly, the novel scaling laws described herein have application for microfluidics, lab on chips, and the like where efficient channels are sought.

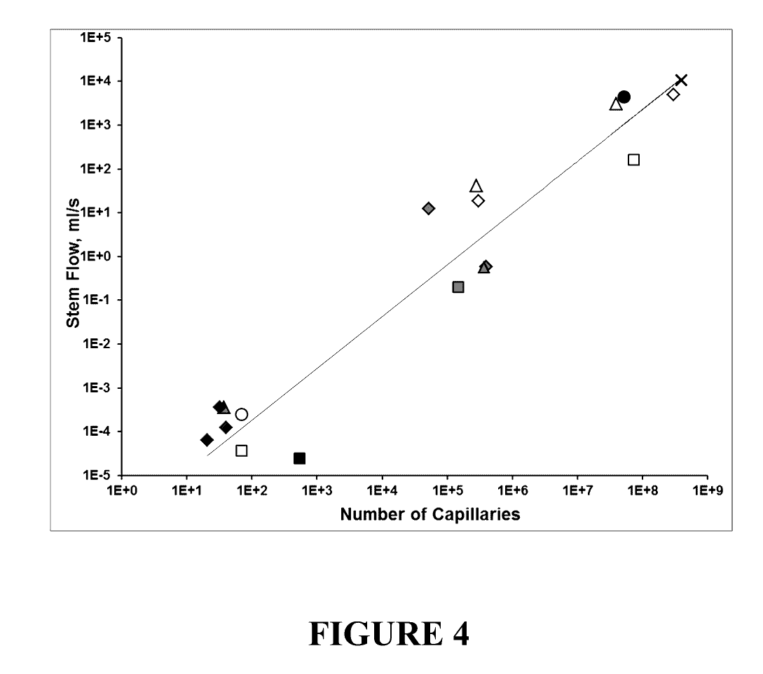

The validated scaling laws disclosed herein also provide a theoretical basis for fundamental studies of drug distribution in various organs. For example, other applications of the disclosed scaling laws relate to drug dose determination with the scaling laws being used to determine a degree of perfusion from biopsy samples. This degree of perfusion can then be used to establish the appropriate dose(s) of drug required, with lower perfusion requiring higher doses and vice versa. Furthermore, as this novel relation holds across species (interspecies, see FIG. 4), the scaling laws can also be used in dosing studies scaled from rodents to larger species and normalized with respect to body weight. Indeed, using the novel scaling laws hereof, the dose can be titrated between species as the number density accurately reflects perfusion (flow per mass) of tissue.

Validation. The predictions of these novel scaling laws were validated using a network flow analysis based on two different models: 1) the asymmetric full model, and 2) the simplified symmetric model (for which there exists morphometric data in the literature (e.g., vessels of various skeletal muscles, mesentery, omentum, and conjunctiva)).

Primarily, the branching pattern and vascular geometry (diameter and length of each vessel segment) of arterial and venous vascular trees of many organs have been measured and are conventionally known. For example, and without limitation, the microvasculature of cat sartorius muscle, hamster retractor muscle, hamster skin muscle, rat mesenteric microvessels, rabbit omentum, and human bulbar conjunctiva microvessels have been reconstructed. Additionally, porcine RCA, LAD, and LCx arterial trees have also been reconstructed and are known, as have human pulmonary arterial and venous trees and a rat pulmonary arterial tree.

Here, for the first model, the entire asymmetric coronary left anterior descending artery (LAD), circumflex branch of the left coronary artery (LCx), and right coronary artery (RCA) trees with several millions of vessels was analyzed. The asymmetric coronary arterial tree was reconstructed in pig hearts by using the growth algorithm introduced by Mittal et al. (N. Mittal et al., A computer reconstruction of the entire coronary arterial tree based on detailed morphometric data, Ann. Biomed. Eng. 33 (8), 1015-1026 (2005a), which is hereby incorporated herein by reference in its entirety) based on the measured morphometric data of Kassab et al. (G. S. Kassab, C. A. Rider, N. J. Tang, Y. C. Fung. Morphometry of pig coronary arterial trees. Am J Physiol. 265(1 Pt 2), H350-65 (1993), which is hereby incorporated herein by reference in its entirety).

Briefly, under laminar and steady flow, the Poiseuille's law for a fluid can be stated as:

$${Q}_{ij} = \frac{π}{128} Δ {P}_{ij} {G}_{ij} \qquad [ 5 ]$$

where Qᵢⱼ is the volumetric flow, in a vessel between any two nodes represented by i and j. ΔPᵢⱼ is the pressure differential given by ΔPᵢⱼ=Pᵢ-Pⱼ, and vessel conductance, Gᵢⱼ, is given by

$${G}_{ij} = \frac{{D}_{ij}^{4}}{{μ}_{ij} {L}_{ij}} d$$

where Dᵢⱼ, Lᵢⱼ, and uᵢⱼ are the diameter, length, and viscosity, respectively, between nodes i and j. The variation of apparent viscosity with vessel diameter is given by A. R. Pries et al., Resistance to blood flow in microvessels in vivo, Circ. Res. 75, 904-915 (1994), which is incorporated herein by reference in its entirety. Two or more vessels emanate from the jth node anywhere in the tree with the number of vessels converging at the jth node being mⱼ. Combining conservation of mass (Eq. [1]) with Eq. [5], obtains a set of linear algebraic equations in pressure for m nodes in the network, namely:

$${\sum }_{i = 1}^{mj} [ {P}_{i} - {P}_{j} ] {G}_{ij} = 0 \qquad [ 6 ]$$

The set of reduce functions was compared to a set of simultaneous linear algebraic terms for the nodal pressures once the conductances were evaluated from the geometry. Additionally, suitable boundary conditions were specified by assigning an inlet pressure of 100 mmHg and a uniform pressure of 25 mmHg at the outlet of the first capillary segment. This system of equations was then solved using a general mean residual algorithm to determine the pressure values at all internal nodes of the arterial tree. The pressure drops as well as the corresponding flows were subsequently calculated.

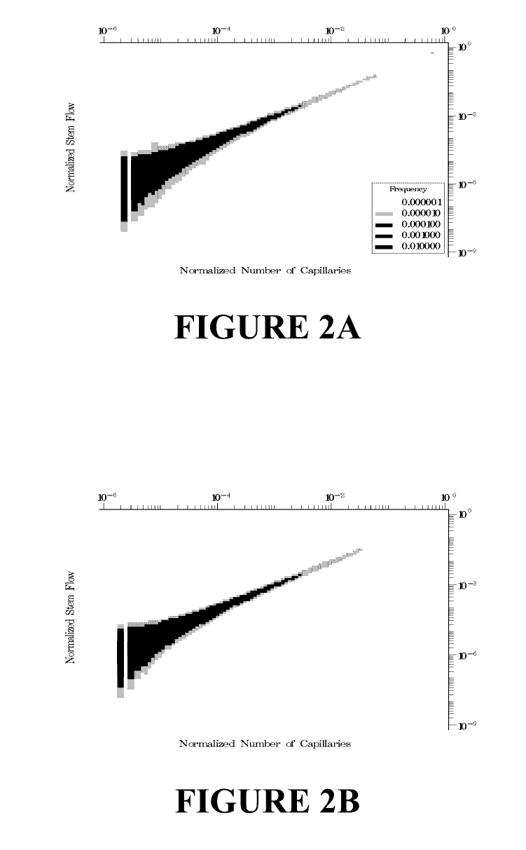

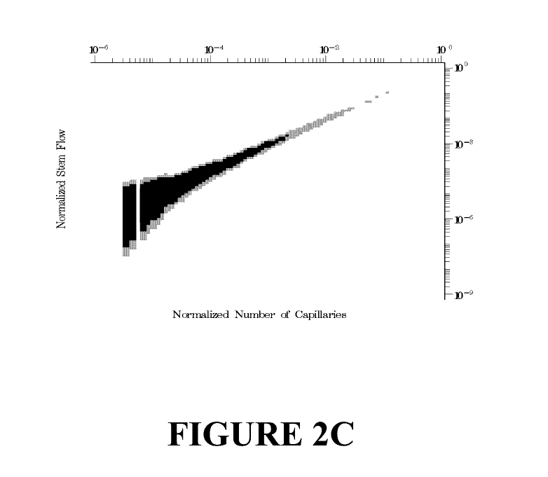

Now referring to FIGS. 2A-2C, log-log density plots of the intraspecific relationships between normalized stem flow and normalized number of capillaries for all stem-crown units of the full asymmetric coronary arterial trees are shown. (The plots show the frequency of data because of the enormity of data points, with darkest shade reflecting highest frequency or density and the lightest shade reflecting the lowest frequency.) The nonlinear regression (SigmaStat 3.5) was used to analyze the data in both asymmetric and symmetric trees, which uses the Marquardt-Levenberg algorithm (nonlinear regression) to find the coefficients (parameters) of the independent variables that give the “best fit” between Eq. [4] and the data. R² represents the correlation coefficient of the power-law fit.

The plots shown in FIGS. 2A-2C illustrate data relate to various arterial trees; namely, the RCA (FIG. 2A), the LAD (FIG. 2B), and the LCx (FIG. 2C) trees. The total numbers of data points shown are 858,353 for FIG. 2A, 936,014 for FIG. 2B, and 572,632 for FIG. 2C, with the solid lines corresponding to least square fits of the inlet flow-number of capillaries data, all of which obey the novel power law relation as described in Eq. [4] with a high correlation coefficient (R²=0.99). Furthermore, the values of λ (Eq. [4]) obtained from FIGS. 2A-2C were 0.93, 0.95, and 0.95 for the porcine RCA, LAD, and LCx, respectively (as compared to a theoretical value of unity, Eq. [3]).

For the second model, a simple symmetric model that simulated the average statistical data of the trees was adapted. Physically, the symmetric model is equivalent to assuming that all the vessel elements in any order or generation are of equal diameter and length and rearranged in parallel, and the blood pressures at all of the junctions between specific orders of vessels are equal (see G. S. Kassab, J. Berkley, Y. C. Fung, Analysis of pig's coronary arterial blood flow with detailed anatomical data. Ann. Biomed. Eng. 25, 204-217 (1997), which is incorporated herein by reference in its entirety).

In this simplified circuit, the flow rate in each element of order n is where Qₘₐₓ/Nₙ, where Qₘₐₓ is the total flow rate into the coronary arterial tree and Nₙ is the total number of vessels at order n. The Qₘₐₓ was determined as the ratio of pressure drop and equivalent resistance of the entire tree. The resistance, R, was computed using Poiseuille's equation

$$R = \frac{128 μl}{π {D}^{4}}$$

(where μ represents the viscosity of blood, and l and D represent the length and diameter of a vessel segment). The equivalent resistance of a crown or the entire tree was then determined by the summation of the vessel segment depending on the series or parallel arrangement.

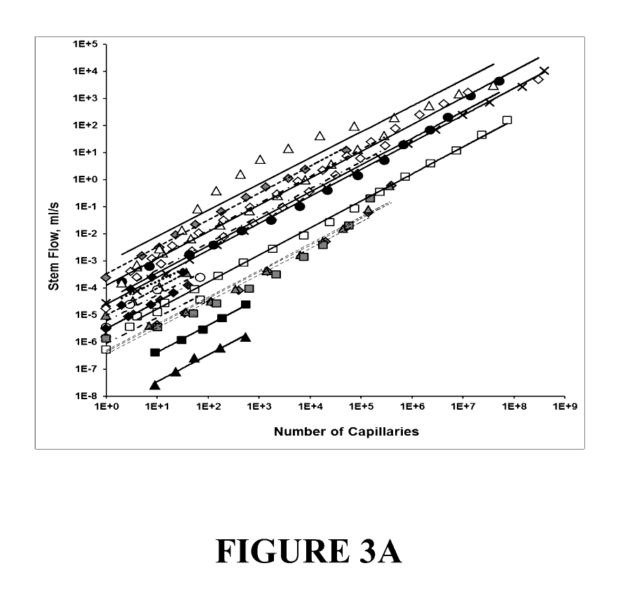

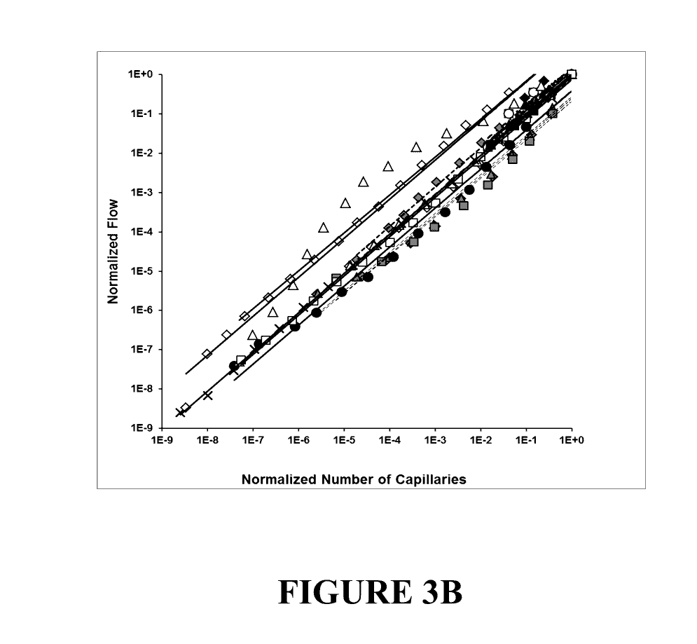

FIGS. 3A and 3B illustrate both absolute and normalized stem flow-crown capillaries for various vascular trees of various species for the symmetric tree analysis (including the coronary arterial tree). As seen, the average data at each order number also obeys a power law relation similar to the asymmetric trees of FIGS. 2A-2C. Table 1 below summarizes the least squares power law relation for each of the vascular trees including the coefficient, exponent, and R² value. The exponents are largely similar to 1 and the R² is highly significant. Additionally, the exponents in the symmetric analysis for the RCA, LAD, and LCx are 0.985, 0.987 and 0.977, respectively, which are notably similar to the results obtained from the asymmetric tree analysis and close to the theoretical unity.

TABLE 1
Intraspecific scaling of stem flow to respective capillary numbers:
Q^{s} ≅ kN_{c}, where Q^{s}, N_{c} and k represent the stem flow, capillary
numbers and proportionality constant, respectively. The power law
equation of the form y = Coefficient x^{Exponent} for each species/organ
is listed. R² represents the correlation coefficient of the power-law fit.

| Species and Organ | Coefficient | Exponent | R² |
|---|---|---|---|
| Hamster Muscle | 3.07E−06 | 1.00 | 1.00 |
| Rat Lungs | 3.23E−04 | 0.998 | 0.996 |
| Rat Mesentery | 9.76E−06 | 1.00 | 1.00 |
| Rabbit Omentum | 2.24E−05 | 0.895 | 0.800 |
| Pig RCA | 4.36E−07 | 0.985 | 0.971 |
| Pig LAD | 4.91E−07 | 0.987 | 0.974 |
| Pig LCX | 3.58E−07 | 0.977 | 0.955 |
| Cat Lungs (arterial) | 4.74E−05 | 0.997 | 0.995 |
| Cat Lungs (venous) | 1.34E−04 | 0.999 | 0.999 |
| Cat Sartorius Muscle (control) | 8.87E−07 | 0.960 | 0.921 |
| Cat Sartorius Muscle | 5.97E−06 | 0.959 | 0.920 |
| Human Skeletal Muscle | 3.09E−06 | 1.00 | 1.00 |
| Human Conjunctiva (arterial) | 3.50E−09 | 0.990 | 0.981 |
| Human Conjunctiva (venous) | 4.21E−08 | 0.998 | 0.996 |
| Human Lungs I (arterial) | 3.73E−05 | 0.985 | 0.986 |
| Human Lungs II (arterial) | 2.42E−05 | 0.999 | 0.999 |
| Human Lungs III (arterial) | 1.23E−04 | 0.992 | 0.984 |
| Human Lungs IV (venous) | 8.87E−04 | 0.963 | 0.928 |
| Human Lungs V (venous) | 1.66E−06 | 0.999 | 0.998 |

In addition to the intraspecific (within species) analysis performed (data shown in FIGS. 2A-3 and Table 1), an interspecific (across species) analysis was also performed. Perhaps more specifically, the inlet flow into the largest stem was plotted against the total number of arterial capillaries for each of the vascular trees of the various species. FIG. 4 shows that the flow-number of capillaries relation still scales as per Eq. [4], with an exponent of 1.18 (R²=0.910), even when applied across species. Accordingly, the aforementioned studies validate the novel form-function scaling laws of the present disclosure that relate to the relation between the capillarity and blood flow, both within and across species.

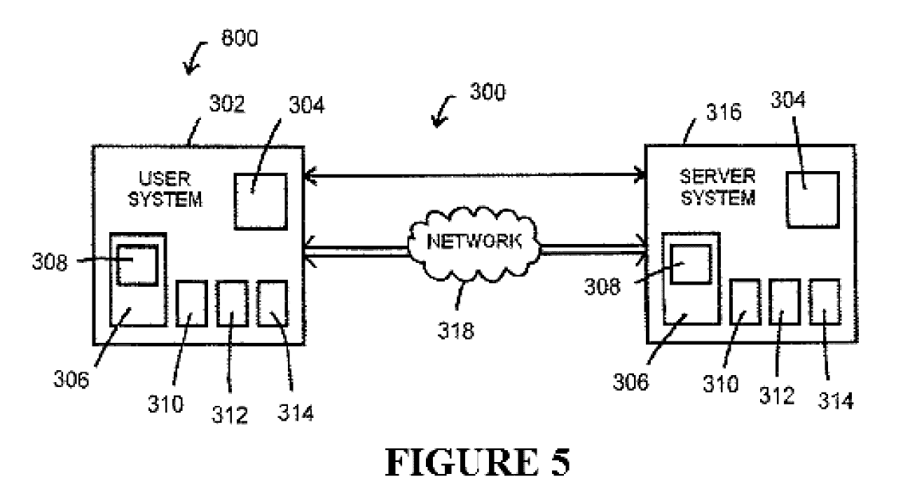

The techniques disclosed herein have tremendous application in a large number of technologies. For example, a software program or hardware device may be developed to diagnose a perfusion inefficiency in a circulatory vessel or organ. At least one exemplary embodiment of such a computer-assisted diagnostic system is shown in FIG. 5. Perhaps more specifically, FIG. 5 shows a diagrammatic view of an embodiment of a diagnostic system 300 comprising a user system 302 and a server system 316. In this exemplary embodiment, user system 302 comprises processor 304 and one or more storage media 306. Processor 304 operates on data obtained by or contained within user system 302. Storage medium 306 may contain, or be in communication with, database 308, whereby database 308 is capable of storing and receiving data. Storage media 306 may contain a program (including, but not limited to, database 308), the program operable by processor 304 to perform a series of steps regarding data relative to vessel and/or tissue measurements (e.g., capillary density measurements) as described in further detail herein.

Any number of storage media 306 may be used with diagnostic system 300 including, without limitation, one or more random access memory, read-only memory, EPROMs, hard disk drives, floppy disk drives, optical disk drives, cartridge media, flash drives, smart cards, and the like, for example. As related to user system 302, storage media 306 may operate by storing data related to vessel and/or tissue measurements for access by a user and/or for storing computer instructions. Processor 304 may also operate upon data stored within database 308.

Regardless of the embodiment of diagnostic system 300 referenced herein and/or contemplated to be within the scope of the present disclosure, each user system 302 may be of various configurations well known in the art and may further comprise one or more data input and/or output devices. By way of example, user system 302, as shown in FIG. 5, comprises keyboard 310, monitor 312, and printer 314. Processor 304 may further operate to manage input and output from keyboard 310, monitor 312, and printer 314. Keyboard 310 is an exemplary input device, operating as a means for a user to input information to user system 302 (for example, and without limitation, patient identification and demographic data). Monitor 312 operates as a visual display means to display data and, in particular, that data related to the vessel and/or tissue measurements and related information. Other input and output devices, such as a keypad, a computer mouse, a fingerprint reader, a pointing device, a microphone, a laser reader, a temporary artery thermometer, and/or one or more speakers are contemplated to be within the scope of the present disclosure. Furthermore, in at least one exemplary embodiment, the user system 302 may comprise an imaging modality configured to obtain a medical image of a patient's vascular network. Additionally or alternatively, the user systems 302 may further comprise one or more communication components such that the processor 304 can receive and/or transmit data to and from its storage media 306 and/or between input/output devices and external devices or databases. For example, in at least one embodiment, the processor 304 may be configured to operate and utilize various wired and/or wireless communication modalities including, without limitation, WiFi, Bluetooth, RFID, radio, and/or any other communication functionality now known in the medical arts or hereinafter developed.

It will be appreciated that processor 304, keyboard 310, monitor 312, printer 314, and other input and output devices and communication components referenced herein may be components of the one or more user systems 302 of the present disclosure.

As previously indicated, the diagnostic system 300 additionally comprises one or more server systems 316. The one or more server systems 316 are in bidirectional communication with the user system 302, either by direct communication (shown by the single line connection on FIG. 5), or through a network 318 (shown by the double line connections on FIG. 5) by one of several configurations known in the art. Such server systems 316 may comprise one or more of the features of a user system 302 as described herein including, without limitation, processor 304, storage media 306, database 308, keyboard 310, monitor 312, and printer 314, as shown in the embodiment of diagnostic system 300 of FIG. 5. Such server systems 316 may allow for bidirectional communication with one or more user systems 302 to allow user system 302 to access data related to a vessel and/or tissue measurement and related information from the server systems 316. It will be appreciated that the user system(s) 302 and/or server system(s) 316 referenced herein may be generally referred to as a “computer.”

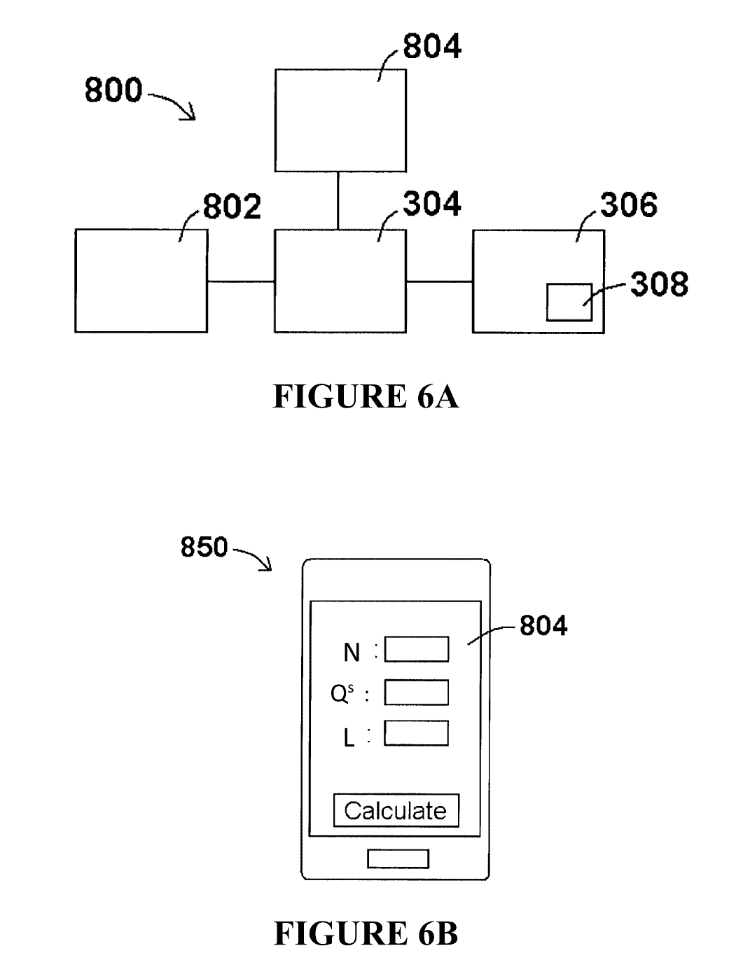

FIGS. 6A and 6B show at least one exemplary embodiment of how the validated scaling laws of the present disclosure can be translated into clinical practice using a data computation system 800 (it will be appreciated that the data computation system 800 of FIGS. 6A and 6B may comprise some, most, or all of the components of previously described diagnostic system 300). In at least one embodiment, exemplary data computation system 800 comprises a processor 304 operably coupled with a storage medium 306 having a program 308 stored thereon. A user interface 802 operably coupled with the processor 304 is configured to receive data indicative of vessel segments and/or a tissue (for example, crown length or capillary density) and display 804—also operably coupled with processor 304—configured to display such vessel segment/tissue data.

Components of various data computation systems 800 of the present disclosure may be contained within, or otherwise part of, computation device 850, such as shown in FIG. 6B. Various computation devices 850 may include, but are not limited to, a desktop computer, a laptop computer, a tablet computer, a portable digital assistant, or a Smartphone. Additionally or alternatively, data computation system 800 may comprise a website, handheld device application, or the like that is configured to provide perfusion or blood flow rates based on a patient's measured or calculated capillary density. Such a website or application can be downloaded to a mobile phone or other portable device, for example, for a quick and easy rule to determine the reference flow for perfusion to quickly, easily, and noninvasively determine if pathology and/or suboptimal perfusion exists in a tissue and/or organ.

In operation, data computation system 800 may be configured such that a user may input data into the device 850 or application for capillary density, stem flow, and/or crown length. Once one of the entries is input, a user clicks the “Calculate” button or otherwise requests computation to yield the capillarity, perfusion, or blood flow rate, depending on the input and consistent with the scaling laws provided herein.

While various embodiments of methods for the noninvasive determination of perfusion and blood flow to a targeted tissue or organ have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments of the present disclosure, the specification may have presented the method and/or process of the present disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present disclosure should not be limited to the performance of their steps in the order written, and one skilled in the art can readily appreciate that the sequences may be varied and still remain within the spirit and scope of the present disclosure.

1. A method for determining perfusion in a mammal, the method comprising the steps of:
determining a capillary density of a targeted tissue comprising at least a portion of a capillary network, the capillary network being part of a mammalian biological tree; and
calculating perfusion of the targeted tissue based on the determined capillary density of the targeted tissue.

2. The method of claim 1, wherein the calculated perfusion is linearly proportional to the capillary density.

3. The method of claim 1, further comprising the step of calculating total flow into the mammalian biological tree based on the determined capillary density of the targeted tissue.

4. The method of claim 3, wherein the calculated total flow is linearly proportional to the capillary density.

5. The method of claim 1, wherein the step of determining a capillary density of a targeted tissue comprises the steps of obtaining a biopsy specimen from the mammal and determining the capillary density of the targeted tissue histologically, wherein the biopsy specimen is representative of the targeted tissue.

6. The method of claim 1, further comprising the step of engineering an artificial tissue comprising a vascular network based on the calculated perfusion.

7. The method of claim 1, wherein the step of determining a capillary density of a targeted tissue comprises the steps of:
using a processor to produce an image showing at least part of the mammalian biological tree proximal to the capillary network of the targeted tissue, wherein the processor is operably connected to a storage medium capable of receiving and storing the image;
identifying a crown length of a vessel portion from the mammalian biological tree image; and
calculating the capillary density of the targeted tissue based on the crown length of the vessel portion.

8. A method for determining a therapeutic drug dosage for a biological subject of a first species, the method comprising the steps of:
determining a capillary density of a targeted tissue, the targeted tissue comprising at least a portion of a capillary network that is part of a mammalian biological tree;
calculating the perfusion of the targeted tissue based on the determined capillary density of the targeted tissue; and
titrating a proper dosage of a therapeutic drug based on the calculated perfusion of the targeted tissue.

9. The method of claim 8, wherein the calculated perfusion of the targeted tissue is linearly proportional to the capillary density of the targeted tissue.

10. The method of claim 8, wherein:
the targeted tissue is a tissue of a biological test subject of a second species;
the method further comprises the steps of:
scaling the proper dosage of the therapeutic drug from the second species to the first species, and
normalizing the scaled dosage with respect to a weight of the biological subject of the first species; and
wherein the first species of the biological subject comprises a human and the second species of the biological test subject is selected from a group consisting of a rodent and a pig.

11. The method of claim 10, wherein instead of calculating the perfusion of the targeted tissue of the biological test subject, the method comprises the step of calculating total flow into the mammalian biological tree based on the determined capillary density of the targeted tissue of the biological test subject, and the step of titrating a proper dosage of a therapeutic drug for a biological subject of a second species is based on the calculated total flow of the targeted tissue of the biological test subject.

12. The method of claim 11, wherein the calculated total flow is linearly proportional to the capillary density.

13. The method of claim 8, wherein the step of determining a capillary density of a targeted tissue is performed histologically.

14. The method of claim 10, wherein the step of determining a capillary density of a targeted tissue comprises obtaining a biopsy specimen from the biological test subject and determining the capillary density of the targeted tissue histologically, wherein the biopsy specimen is representative of the targeted tissue of the biological test subject.

15. A method of identifying a deviation in perfusion rate in a mammal, the method comprising the steps of:
establishing a baseline capillary density from a plurality of healthy mammals of a first species;
establishing a calculated baseline perfusion rate based on the baseline capillary density;
determining a capillary density of a test subject; and
comparing the capillary density of the test subject to the baseline capillary density, wherein a deviation from the baseline capillary density is indicative of the test subject having a perfusion rate that is not in accordance with the baseline perfusion rate.

16. The method of claim 15, wherein the calculated baseline perfusion rate is linearly proportional to the baseline capillary density.

17. The method of claim 15, wherein the test subject comprises a mammal of a second species.

18. The method of claim 15, wherein the step of determining a capillary density of a test subject is performed histologically.

19. The method of claim 15, wherein the step of determining a capillary density of a test subject is performed using an image of a capillary network that is part of a biological tree of the test subject.

20. The method of claim 15, further comprising the steps of:
identifying that the capillary density of the test subject deviates from the baseline capillary density;
calculating a perfusion rate of a test subject based on the capillary density of the test subject; and
assigning a diagnosis to the test subject based on the calculated perfusion rate;
wherein the calculated perfusion rate of the test subject is linearly proportional to the capillary density of the test subject.
